(19) United States
(12) Patent Application Publication
(10) Pub. No.: US 2026/0184657 A1
(43) Pub. Date: Jul. 2, 2026

(54) FURNACE

(71) Applicant: INOVYN EUROPE LIMITED, Runcorn, Cheshire (GB)

(72) Inventors: Paul DEGRAEVE, Isieres (BE); Roel MILIS, Mechelen (BE)

(21) Appl. No.: 19/130,322

(22) PCT Filed: Nov. 16, 2023
(86) PCT No.: PCT/EP2023/082080

§ 371 Date: May 15, 2025

(30) Foreign Application Priority Data
Nov. 22, 2022 (EP) 22208936.9

(51) Int. Cl. C07C 17/25; B01J 6/00; F27D 11/00; H05B 3/62

(52) U.S. Cl. CPC C07C 17/25; B01J 6/00; F27D 11/00; H05B 3/62; F27M 2003/16; H05B 2203/016

(57) ABSTRACT

An electrically heated furnace, particularly a furnace for chemical reaction, the furnace includes a furnace cabin that includes one or more process tubes which extend through the furnace cabin and a plurality of electrically powered heating means located inside the furnace cabin for heating the process tubes, as well as electricity supply means located outside the furnace cabin for providing electricity to the electrically powered heating means. The electricity supply means includes one or more hermetically sealed boxes, connected externally to the furnace cabin, but spaced away therefrom, and the electrically powered heating means are connected to an electricity supply within the hermetically sealed box by one or more electrical connecting rods which pass through the wall of the hermetically sealed box and through one or more hermetically sealed electrical feed-throughs. A process for performing a chemical reaction in the furnace is also provided.

### FIELD AND BACKGROUND OF THE INVENTION

The present invention relates to an electrically heated furnace (and which as used herein may also be referred to as a reactor), particularly a furnace for chemical reaction, for example for cracking of hydrocarbons such as ethane, naphtha and 1,2-dichloroethane. The present invention also relates to an electrically heated furnace used for heating-up petroleum or chemical products or for gas processing.

Furnaces for cracking of hydrocarbons such as ethane, naphtha, 1,2-dichloroethane and others are well-known. Historically, the reaction has been performed by passing the reactant hydrocarbon through an externally heated reaction tube located within a furnace to provide the heating for the endothermic cracking reaction. In traditional processes the heat required has been obtained by combustion of a fuel in burners located inside the furnace.

It has also been proposed to provide the heat by electrical heating, for example using heating elements located on the walls of the furnace or heating coils on the reaction tubes. Also direct electrical heating of the reaction tubes has been proposed.

An issue with electrical heated furnaces is that this requires provision of electrical energy into a furnace which is not only at high temperatures, but where the external environment outside of the furnace is an hazardous “ATEX” area. “ATEX” generally refers to an area in which there is, or is a risk of, the presence of flammable gases which can give rise to the risk of explosion. Thus, any sources of ignition must be avoided.

It is known to provide sealed boxes for electrical connections in ATEX areas. However, a particular issue arises in the case of electrically heated furnaces because of the very high temperatures inside the furnace during operation, of the high electrical power input and of the requirement to transfer the electrical power through the wall of the furnace.

Connecting rods are commonly used to transfer electrical power through the wall of the furnace. When the furnace is located in an ATEX area, the issue is to find a secure and reliable way to connect the rods to the electrical feeding cable.

In WO 2021214256 the issue of electrical connections to a cracking furnace is addressed by using a purged connection box and a gas permeable connection to the furnace. In particular, an inert gas is fed to a connection box, and then passes to the furnace. The flow of inert gas prevents outwards flow of gas from the furnace. In such a system, however, the furnace is effectively still in “open connection” to the connection box, and hence this requires significant monitoring. In particular, on expansion to reaction conditions it is still difficult to ensure a reasonably good seal, and to ensure flow through all “openings” is prevented, so it becomes necessary to ensure a significant inert gas flow is maintained. In case of loss of inert gas flow, external contaminated air can enter the connection box or alternatively, gas from inside the furnace may enter into the connection box. Also, the loss of inert gas flow to any box will necessitate complete furnace shut-down.

WO 2021180864 and its US counterpart, US 2023116690, also describe a reactor vessel suitable for steam cracking with a connection chamber for the electrical connection of the heating rods. The connection chamber, as in WO 2021214256, is connected directly to the reactor vessel and designed to be gas permeable to the reactor vessel.

### SUMMARY OF THE INVENTION

We have developed an improved system in which the electrical feed connections are completely hermetically isolated from the furnace and also from the external environment. In particular, the present invention provides an electrical connection arrangement that is suitable to be operated in a dangerous flammable area (ATEX area).

Thus, in a first aspect, the present invention provides an electrically heated furnace, the furnace comprising:
a. A furnace cabin, the furnace cabin comprising:
   one or more process tubes which extend through the furnace cabin and
   a plurality of electrically powered heating means located inside the furnace cabin for heating the one or more process tubes,
b. Electricity supply means located outside the furnace cabin for providing electricity to the electrically powered heating means,
characterised in that
   a. The electricity supply means comprises one or more hermetically sealed boxes, connected externally to the furnace cabin, but spaced away therefrom,
   b. the electrically powered heating means are connected to an electricity supply within the hermetically sealed box by one or more electrical connecting rods which pass through the wall of the hermetically sealed box, the rods passing through a wall of the hermetically sealed box through one or more hermetically sealed electrical feed-throughs.

The present invention provides an electrically heated furnace.

The furnace comprises a furnace cabin, which comprises one or more process tubes which extend through the furnace cabin. In general, in use, reactants to be reacted are passed through the inside of the process tubes. The process tube or tubes are heated, and thereby the reactants or a process fluid passing through the process tube or tubes are heated and caused to react.

Furnaces of this general structure, albeit using other heating means, are known for a variety of different chemical processes, including for heating-up petroleum or chemical products or for gas processing. The process tube or tubes may, for example, each be a single straight tube which passes once through the furnace cabin. Alternatively, the process tube or tubes may, for example, have several straight sections within the furnace, linked by elbows or 180° bends e.g. to form a serpentine structure.

Particularly useful furnaces for the process of the present invention are furnaces for cracking of hydrocarbons such as ethane, naphtha and 1,2-dichloroethane.

Conventional furnaces for such reactions utilise burners located on the walls of the furnace cabin which burn a fuel to generate the heating required for heating the process tube or tubes. In the present invention the process tube or tubes are electrically heated. In particular, there are provided a plurality of electrically powered heating means located inside the furnace cabin for heating the one or more process tubes. Any suitable heating means may be provided. In one embodiment, the plurality of electrically powered heating means may be a plurality of electrically powered heating elements, in particular located on the walls inside the furnace cabin. In another embodiment, the plurality of electrically powered heating means may be a plurality of heating coils, in particular each being provided in the furnace cabin adjacent to one or more process tubes. In another embodiment, the plurality of electrically powered heating means may be means by which a process tube or tubes are directly electrically heated. Combinations of different heating means may also be used, including combinations of the above examples.

The furnace also comprises an electricity supply means located outside the furnace cabin for providing electricity to the electrically powered heating means.

In particular, the present invention provides electrical connections inside of one or more hermetically sealed boxes (and which as used herein may also be referred to as “ATEX boxes”) which boxes are connected externally to the furnace cabin, but spaced away therefrom. In particular, the connection to the heating means is provided by electrical connecting rods which pass through the wall of the hermetically sealed box, the rods passing through the wall of the hermetically sealed box through one or more hermetically sealed electrical feed-throughs.

The term “hermetically sealed” as used herein, and as would be understood by the person skilled in the art more generally, means that the hermetically sealed box is completely gas tight. Thus, gas does not flow out of or in to the hermetically sealed box (once it has been sealed).

Suitable hermetically sealed electrical feed-throughs are known for use in other areas. Examples include PAVE seals (Pressure and Vacuum Electrical Terminal Seals) of PAVE Technology Co.® or bushing conductor studs of BARTEC or STUDSEAL HIGH-CURRENT FEEDTHROUGHS of DOUGLAS Electrical Components. Typically these use one or more epoxy seals or ceramic seals or glass seals. These may be rated at up to 200° C.

To ensure that such seals are suitable in the present invention, therefore, the one or more hermetically sealed boxes are spaced away from the outside of the furnace cabin. For example, the side/wall of the hermetically sealed box facing the outside wall of the furnace cabin may be from 5 to 70 cm away from the outside wall of the furnace cabin; more preferably from 10 to 50 cm away from the outside wall of the furnace cabin. This may be achieved, for example, by use of a suitable spacer box or brackets or by use of other connecting means which maintain the required distance from the wall of the furnace cabin. The appropriate space between the one or more hermetically sealed boxes and the furnace cabin wall can be selected by the person skilled in the art, but is generally dependant of the temperature inside of the furnace cabin and of the characteristics (current, voltage) of the power transferred via the connection rods to the electrical heating means inside the furnace cabin.

Inside the furnace cabin, the temperature is very high, typically from 500 to 1300° C. depending on the application. Although insulation layers are installed on the inside wall of the furnace cabin, heat is transferred to the furnace cabin wall. Typical furnace cabin wall temperatures are well above ambient temperature.

Heating means and connection rods inside the furnace are exposed to the high temperatures. There is heat transfer by the connection rods by conduction, whilst electrical currents also generate heat in the connection rods by Joule effect. Both lead to increases of the temperature of the connection rod. (Any suitable conductive metals may be used for the connection rods. However, at least in or close to the furnace the connecting rods are preferably formed of materials which can withstand high temperatures on a repeated or extended basis. Examples include alloys, such as nickel-chromium (NiCr) alloys or iron-chromium-aluminium (FeCrAl) alloys or molybdenum disilicide (MoSi₂) alloys.

By spacing the hermetically sealed box away from the wall of the cabin furnace, the temperature of the extremity of connection rods, towards the hermetically sealed boxes, is reduced, allowing the electrical connection with the electrical supply to be at a location of moderate temperature, acceptable for the cables and for the hermetically sealed boxes.

A particular advantage of the present invention is that it is not necessary to provide cooling to the hermetically sealed box. (For avoidance of doubt, it would still be possible to provide cooling to the hermetically sealed box. However, this provides additional complexity when not needed. Preferably therefore no cooling means are provided for the hermetically sealed box.) In the most preferred embodiments of the present invention the one or more hermetically sealed boxes are spaced away from the outside of the furnace cabin by use of a suitable spacer box or boxes. The term “spacer box” as used in this context means that the space between the furnace cabin and the hermetically sealed box is itself enclosed (or “boxed in”). In preferred embodiments the spacer box or boxes will be gas tight to the external environment/atmosphere. However, it/they need not be gas tight to the furnace cabin atmosphere. During use in preferred embodiments the atmosphere inside the spacer box is the same as that inside the furnace cabin (external to the process tubes). (In general, in use of the furnace there is provided in the furnace cabin a suitable gaseous atmosphere. Typically an inert or largely inert atmosphere is preferred, although a small amount of oxygen may be desirable for the use of certain types of heating elements. This is described further below.) It may be noted that, whilst it is preferred that no cooling means are provided for the hermetically sealed box, the connecting box may be cooled, for example, provided with cooling panels through which a cooling fluid can flow.

In one embodiment, the outside wall of the furnace cabin and the side/wall of the hermetically sealed box through which the connecting rods pass will be substantially parallel to each other, and the spacing between them is therefore the perpendicular distance between them. (Typically the outside wall of the furnace cabin and the side/wall of the hermetically sealed box will both be essentially vertical, and the distance will then be the horizontal distance.) Where the planes are not substantially parallel, then the spacing should be measured as the perpendicular distance between the outside wall of the reactor cabin and the location where the connecting rods exit the hermetically sealed box.

An advantage of this embodiment is that connecting rods may be used where a single rod passes through both the wall of the furnace cabin and the wall of the hermetically sealed box. (And in preferred embodiments with a spacer box the connecting rods also pass through the spacer box in between these.) An example of this is shown in FIGS. 2 and 4 as discussed further below.

In another embodiment the hermetically sealed box may be orientated such that the wall of the hermetically sealed box through which the one or more connecting rods pass (and hence in which the one or more hermetically sealed electrical feed throughs are present) is at an angle, for example 45 to 90 degrees, and more preferably such as 90 degrees, to the wall of the furnace cabin. For example, there may be provided a first set of one or more connecting rods which pass through the one or more hermetically sealed electrical feed throughs of the hermetically sealed box, which in turn are connected to a second set of one or more connecting rods which pass through the wall of the furnace cabin (and then connect to the heating means). In particularly preferred embodiments the connecting rods of the first set are connected to connecting rods of the second set inside a spacer box. Connecting rods of the first set may be connected to connecting rods of the second set by flexible connections which enable the two sets of connecting rods to not be axial. An example of this is shown in FIG. 3 which will be discussed further below. A particular advantage of this embodiment is that relatively short connecting rods can be used for either set, particularly the first set. This can make expansion of the connecting rods on heating easier to deal with. (As well as providing a better seal of the first set because expansion is “less”, an advantage in relation to the second set of connecting rods is that they may be fixed in place at the wall of the furnace cabin, and do not need to be able to move to accommodate expansion.) In one embodiment, the length of the connecting rods of the first set is less than 40 cm and/or the length of the connecting rods of the second set, is less than 70 cm in length. Also, the connecting rods can be of different materials. For example, connecting rods for passing through the one or more hermetically sealed electrical feed throughs may be a cheaper material, such as copper, whilst the material for the second set may be selected from materials which have low coefficients of expansion and/or which are otherwise preferred for connection to the heating elements within the furnace (such as iron-chromium-aluminium (FeCrAl) alloys). (For avoidance of doubt, connecting rods with sections of different materials can also be used with “straight” configurations such as in FIGS. 2 and 4. For example, a cheaper material, such as copper, may be used for the section at the one or more hermetically sealed electrical feed throughs and another material, such as a FeCrAl alloy, used for the section closer to or within the furnace) A further advantage of smaller connecting rods and the angled orientation is that it is easier to access the space between the hermetically sealed box and the furnace, for example to replace one or more connecting rods or connectors.

### BRIEF DESCRIPTION OF DRAWINGS

Some examples of particularly preferred configurations are shown in the following Figures.

FIG. 1 is a schematic depiction of a furnace.

FIG. 2 is a “close-up” depiction of a hermetically sealed box connected to the furnace cabin.

FIG. 3 is a “close-up” depiction of an alternative configuration for connecting a hermetically sealed box to the furnace cabin.

FIG. 4 is a “close-up” depiction of another configuration for connecting a hermetically sealed box to the furnace cabin.

### DETAILED DESCRIPTION OF THE INVENTION

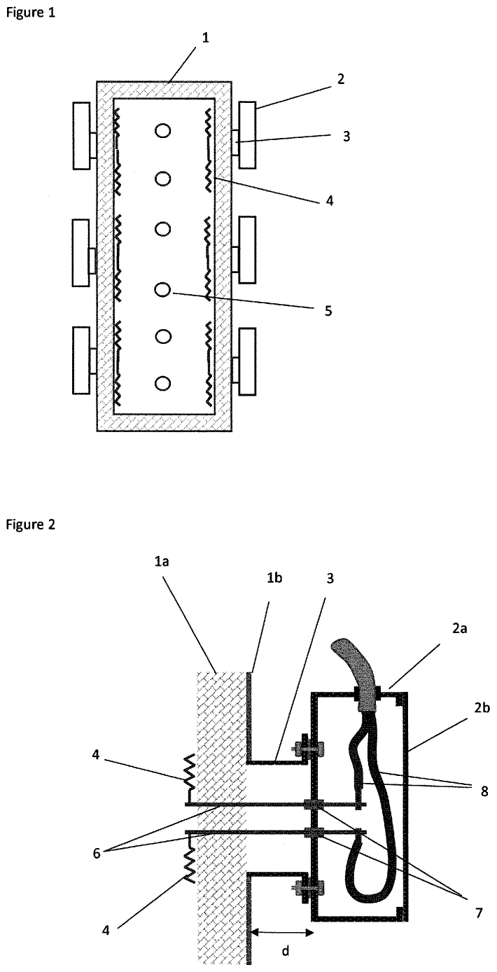

FIG. 1 shows schematically a furnace comprising a furnace cabin (1) with hermetically sealed boxes (2) connected to the outside wall of the furnace cabin via spacers boxes (3). Inside the furnace cabin are provided a plurality of heating elements (4) for heating process tubes (5). It is noted that this drawing is provided for simple illustration purposes only and no particular heed should be taken of the relative scales or number of particular features. For example, as shown there are six hermetically sealed boxes and also six process tubes, but in practise many more of each may be present, whilst the boxes (2) will be much smaller relative to the furnace cabin (1).

FIG. 2 shows a “close-up” of one of the hermetically sealed boxes connected to the furnace cabin. In FIG. 2, there can be seen that the wall of the furnace cabin has a layer of insulating material (1a) surrounded by an outer wall (1b), and the hermetically sealed box has a main body (2a) and a hermetically tight lid (2b). As shown in FIG. 2, the hermetically sealed box is connected to a spacer box (3) which extends from the furnace cabin wall, giving a separation distance “d”. (As shown the hermetically sealed box is bolted to the spacer box (3) with bolts and gaskets (not labelled) to give a seal between the inside volume of the spacer box (3) and the external atmosphere.) Also shown in FIG. 2 are two connecting rods (6) which are connected to heating elements (4), pass through the wall of the furnace cabin and through the wall of the hermetically sealed box via hermetically sealed electrical feed-throughs (7), and connect to electricity supply cables (8). Note here that this number of rods is indicative, and need not be two.

Due to the distance “d”, the temperature of the hermetically sealed electrical feed-throughs (7) can be maintained significantly below the temperature of the outside wall of the furnace cabin, enabling a reliable hermetic seal to be maintained.

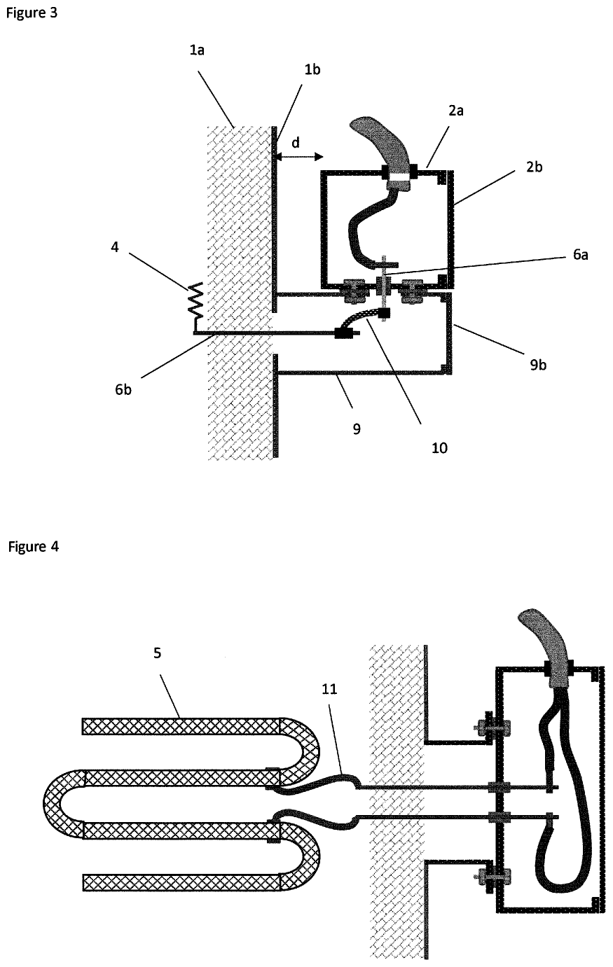

FIG. 3 shows a “close-up” of an alternative configuration for connecting a hermetically sealed box to the furnace cabin. In FIG. 3, there can again be seen that the wall of the furnace cabin has a layer of insulating material (1a) surrounded by an outer wall (1b). As shown in FIG. 3, the hermetically sealed box, having a body (2a) and a lid (2b) is connected to a spacer box (9) which extends from the furnace cabin wall, giving a separation distance “d”. This spacer box has a hermetically tight lid (9b) which can be opened to allow access. Also shown in FIG. 3 is a first connecting rod (6a) connected, via a flexible connection (10) to a second connecting rod (6b) to provide the overall electrical connection between the electricity supply cable in the hermetically sealed box and the electrical heating means (4). Note again that the number of rods in the first and second set is indicative, and need not be one in each set as shown. It is also possible, for example, to connect a single first rod to two or more second rods or to connect several first rods to several second rods.

A particular advantage, as noted, is that this configuration allows to provide a spacer box (9) with hermetically sealed lid (9b) which can be used to easily access the connecting rods and the hermetically sealed feed throughs.

FIG. 4 shows a further example of the present invention. In this case the furnace cabin (1), hermetically sealed box (2) and other components are largely as for FIG. 2 except that heating elements (4) are not present on the inside wall of the furnace cabin. Instead, the process tubes (5), shown schematically in FIG. 3, are heated directly, and the connecting rods are connected directly to the process tubes (5) by flexible connectors (11).

The furnace of the present invention may be used for any process which typically operates in furnaces in which reactants or process fluids are passed through heated process tubes, including any processes conventionally or historically operated in fired (burner based) furnaces.

Thus, in a second aspect, the present invention provides a process for performing a chemical reaction which process comprises
a. providing a furnace according to the first aspect of the invention,
b. passing one or more reactants through the one or more process tubes, and
c. heating the reactants using the electrically powered heating means to effect reaction of the reactants within the process tubes.

The temperature of the reaction will depend on the specific process and is not especially limited, but in preferred processes the furnace may typically operate at a temperature of the process tubes in the range 300 to 1200° C.

The process may be catalytic or non-catalytic. In the former case, catalysts may be provided in the process tubes either as a catalyst bed or as a coating on the inside of the process tubes.

Examples of suitable processes include steam and other cracking processes, various reforming processes, such as steam reforming and dry reforming, processes for the dehydrogenation of alkanes.

In particularly preferred embodiments, the process is a process for cracking, and most particularly a process for cracking of 1,2-dichloroethane (EDC) to produce vinyl chloride monomer (VCM). The cracking of EDC to produce VCM is well-known in the art. The present invention, similar to operation in a conventional process operates in a furnace by passing an EDC containing stream through a process tube inside the furnace cabin, and heating the tube to heat and crack the EDC therein.

Typically, and preferably, the (or each if more than one) process tube in such a furnace is in the form of a serpentine tube which is located in a vertical plane or close to a vertical plane in the centre of the furnace cabin. Heating can then be applied from heating elements on both sides of the furnace cabin. This is, for example, shown in concept in FIG. 1 where the process tubes may be considered to represent the horizontal sections of a serpentine tube.

The EDC containing stream is heated to a temperature sufficient to cause cracking of the EDC. Typically this is at least 350° C., and preferably in the range 350° C. to 550° C. The temperatures, residence times etc. may be selected by the person skilled in the art for the degree or rate of cracking required. They may, in particular, be similar to cracking in conventional (hydrocarbon burner) systems.

The EDC containing stream may be introduced in a form where the EDC is in the liquid phase. In this case the EDC is vaporised in the earlier sections of the process tube or tubes, then further heated to a temperature, for example within the range 350° C. to 550° C., at which cracking occurs in later sections. In other embodiments the EDC containing stream introduced at the inlet may have been heated (“pre-heated”) externally to the furnace, for example to a temperature sufficient to vaporise any liquid EDC. In such a case the EDC containing stream is in the gaseous phase at the inlet of the process tube(s).

The furnace may however also be designed for and used for other processes, such as steam cracking. The furnace may also be designed for heating-up petroleum or chemical products.

As previously noted, during the process there may be provided in the furnace cabin a suitable gaseous atmosphere. In general an inert or largely inert atmosphere is preferred, although a small amount of oxygen may be desirable for the use of certain types of heating elements. The furnace chamber may be a closed system, by which is meant that under normal operation gas does not enter or exit in any significant quantity, or may comprise means to operate a steady flow of gas, such as an inert gas or a gas mixture comprising an inert gas, into and out of the chamber. An advantage of this latter system is that the outflowing gas can be analysed to check for any contaminants that might indicate a leak e.g. of reactants from process tubes or air from outside the furnace.

The furnace cabin may also, or alternatively, be held at an elevated pressure. This is advantageous in relation to potential external leaks because it ensures that the gas in the furnace cabin leaks “out” rather than air leaking “in”.

In the most preferred embodiments of this second aspect the one or more hermetically sealed boxes are spaced away from the outside of the furnace cabin by use of a suitable spacer box or boxes as already discussed for the first aspect. In preferred embodiments the spacer box or boxes will be gas tight to the external environment/atmosphere but not to the furnace cabin atmosphere. Thus, during the process of this second aspect of the invention the atmosphere inside the spacer box is the same as that inside the furnace cabin (external to the process tubes).

In particularly advantageous embodiments the atmosphere in the spacer box is not only the same as that in the furnace cabin, but so is the pressure, and both the spacer box and the furnace cabin are at elevated pressure. This ensures also that gas in the spacer box leaks “out” rather than air leaking “in”.

The present invention has the advantage to minimize the amount of inert gas required for the electrical connection system. This can also minimize the gas flow that exit the furnace cabin and the associated heat losses.

### EXAMPLE

An electrical heater/spaced hermetically sealed box system has been modelled with computational fluid dynamics (CFD). The system is equivalent to FIG. 2 except that a connecting rod (6) passes from the hermetically sealed box (2) to a single heating element (4) inside the furnace cabin (1). The distance, d, is 500 mm. The wall inside of the furnace cabin has 300 mm of insulation (1a). The connecting rod is 20 mm in diameter along its length, the first 400 mm of the connecting rod (at the furnace end, and passing through the furnace wall) is a FeCrAl alloy whilst the rest is copper.

A current of 262.5 A is passed to the heating element from an electricity supply in the hermetically sealed box and through the connecting rod. The temperature inside the furnace cabin is 816° C. The connecting rod at the junction of the FeCrAl section and the copper section (at a point outside but close to the wall of the furnace cabin) is at approximately 135° C. At the wall of the spacer box i.e. 50 cm from the outside of the furnace cabin, the connecting rod is at a temperature of approximately 60° C. This temperature is suitable for the use of PAVE seals of PAVE Technology Co. for passage of the connecting rod through the wall of the hermetically sealed box.

1. An electrically heated furnace, the furnace comprising:
a. A furnace cabin, the furnace cabin comprising:
i. one or more process tubes which extend through the furnace cabin and
ii. a plurality of electrically powered heating means located inside the furnace cabin for heating the one or more process tubes,
b. Electricity supply means located outside the furnace cabin for providing electricity to the electrically powered heating means, wherein
a. the electricity supply means comprises one or more hermetically sealed boxes, connected externally to the furnace cabin, but spaced away from the furnace cabin, and
b. the plurality of electrically powered heating means are connected to an electricity supply within the one or more hermetically sealed boxes by one or more electrical connecting rods which pass through a wall of the one or more hermetically sealed boxes, the one or more electrical connecting rods passing through the wall of the one or more hermetically sealed boxes through one or more hermetically sealed electrical feed-throughs.

2. The electrically heated furnace according to claim 1, wherein the plurality of electrically powered heating means includes at least one of the group consisting of: a plurality of electric heating elements, a plurality of electric heating coils, and a plurality of means for direct electrical heating of the process tube or tubes.

3. The electrically heated furnace according to claim 1, wherein the one or more hermetically sealed boxes are spaced away from an outside of the furnace cabin by use of one or more spacer boxes.

4. The electrically heated furnace according to claim 3, wherein the one or more spacer boxes are gas tight to an external environment/atmosphere.

5. The electrically heated furnace according to claim 3, wherein the one or more spacer boxes are not gas tight to the furnace cabin, such that during use an atmosphere inside the one or more spacer boxes is the same as an atmosphere inside the furnace cabin.

6. The electrically heated furnace according to claim 1, wherein the one or more hermetically sealed boxes are orientated such that the wall of the one or more hermetically sealed boxes through which the one or more connecting rods pass, and hence in which the one or more hermetically sealed electrical feed throughs are present, is at an angle to a wall of the furnace cabin.

7. The electrically heated furnace according to claim 6, wherein there is provided a first set of one or more connecting rods which pass through the one or more hermetically sealed electrical feed throughs of the one or more hermetically sealed boxes, which in turn are connected to a second set of one or more connecting rods which pass through the wall of the furnace cabin.

8. The electrically heated furnace according to claim 7, wherein the one or more connecting rods of the first set are connected to the one or more connecting rods of the second set inside a spacer box.

9. The electrically heated furnace according to claim 7, wherein a length of the one or more connecting rods of the first set is less than 40 cm and/or a length of the one or more connecting rods of the second set is less than 70 cm.

10. The electrically heated furnace according to claim 7, wherein the one or more connecting rods of the first set and the one or more connecting rods of the second set are made of different materials.

11. A process for performing a chemical reaction, which process comprises:
a. providing a furnace according to claim 1,
b. passing one or more reactants through the one or more process tubes, and
c. heating the one or more reactants using the electrically powered heating means to effect reaction of the one or more reactants within the one or more process tubes.

12. The process according to claim 11 wherein the furnace operates at a temperature of the one or more process tubes in a range of 300 to 1200° C.

13. The process according to claim 11 wherein the process is a process for cracking of 1,2-dichloroethane (EDC) to produce vinyl chloride monomer (VCM), and wherein an EDC containing stream is heated in the one or more process tubes to a temperature in a range of 350° C. to 550° C.

14. The process according to claim 11 wherein there is provided in the furnace cabin an inert or largely inert gaseous atmosphere.

15. The process according to claim 14 wherein the furnace cabin is kept above atmospheric pressure.

16. The electrically heated furnace according to claim 1, wherein a wall of the one or more hermetically sealed boxes facing an outside wall of the furnace cabin is from 5 to 70 cm away from the outside wall of the furnace cabin.
